(19) United States
(12) Patent Application Publication
(10) Pub. No.: US 2025/0327401 A1
(43) Pub. Date: Oct. 23, 2025

(54) METHOD FOR CHARACTERIZING INTER-WELL CONNECTIVITY, DEVICE, MEDIUM AND PRODUCT

(71) Applicant: INSTITUTE OF GEOLOGY AND GEOPHYSICS, CAS, Beijing (CN)

(72) Inventors: Yuran Zhang, Beijing (CN); Tianming Huang, Beijing (CN); Shengwen Qi, Beijing (CN)

(21) Appl. No.: 18/790,796

(22) Filed: Jul. 31, 2024

(30) Foreign Application Priority Data
Apr. 18, 2024 (CN) 202410472021.7

(51) Int. Cl. E21B 49/08; C12Q 1/6806; C12Q 1/6874; C12Q 1/689; G16B 20/20; G16B 30/00

(52) U.S. Cl. CPC E21B 49/08; C12Q 1/6806; C12Q 1/6874; C12Q 1/689; G16B 20/20; G16B 30/00

(57) ABSTRACT

Provided are a method for characterizing inter-well connectivity, a device, a medium, and a product. The method includes: collecting a fluid microbial sample from an injection well and each of the production wells in a selected well field; subjecting each fluid microbial sample to 16S rRNA gene amplicon sequencing to obtain raw sequencing data of the injection fluid and raw sequencing data of each production fluid; extracting amplicon sequence variants (ASVs) from the raw sequencing data of the injection fluid and the raw sequencing data of each production fluid to obtain an ASV table of the injection fluid and an ASV table of each production fluid; and subjecting the ASV tables of each injection-production well pair to two-circle Venn diagram analysis to obtain the number of ASVs overlapped for each injection-production well pair, and determining inter-well connectivity between each injection-production well pair accordingly.

### CROSS REFERENCE TO RELATED APPLICATION

This patent application claims the benefit and priority of Chinese Patent Application No. 2024104720217, filed with the China National Intellectual Property Administration on Apr. 18, 2024, the disclosure of which is incorporated by reference herein in its entirety as part of the present application.

### TECHNICAL FIELD

The present disclosure relates to the field of subsurface energy and resource extraction, and in particular to a method for characterizing inter-well connectivity based on the natural microbial community signal in an underground reservoir, a device, a medium, and a product.

### BACKGROUND

During the development of underground resources, such as oil and gas extraction, water resource extraction, underground energy storage, carbon dioxide geosequestration, geothermal energy extraction, among other scenarios, it is often necessary to inject water into deep reservoirs to maintain formation pressure, enhance oil and gas recovery, extract or exchange heat, and store energy/resource. Characterizing the inter-well connectivity between an injection well and a production well during a reservoir injection-production process is an important step for the efficient development and effective management of subsurface resources, and is also a prerequisite for reservoir modeling and production forecasting.

Because the permeability structure of the inter-well region of a reservoir is unknown, heterogenous, and subjected to changes over time, the connectivity between an injection well and each production well is often difficult to determine and needs to be characterized using artificial tracer technology. Artificial tracer technology refers to the following process: a marker (namely, a tracer) is added to an injection well such that the marker enters the reservoir and travels with the reinjected fluid, and it is continuously sampled and monitored in a production well to obtain the change in tracer concentration over time (namely, a breakthrough curve). A tracer mass recovery can be obtained by fitting and integrating over the breakthrough curve. Tracer mass recovery in each production well reflects the relative connectivity of each production well with the injection well. However, a tracer can only be pulse-injected during a field tracer experiment instead of continuously injected, and the tracer is usually heavily diluted by formation water. As a result, an injected tracer often cannot be captured in a production well, or only produces noisy data without any interpretable breakthrough curve, leading to high failure rates for field tracer experiments. Furthermore, each tracer experiment requires high-frequency and continuous monitoring of an observation well, and the field sampling often needs to continue for weeks or even months to obtain a complete breakthrough curve, resulting in high logistical costs. Finally, tracers are mostly chemical compounds, which will contaminate formation water after being injected into the formation. The above problems severely limit the applicability of artificial tracer experiments.

### SUMMARY

The objective of the present disclosure is to provide a method for characterizing inter-well connectivity, a device, a medium, and a product. Utilizing the fact that exogenous injection water and produced formation water often bear distinct microbial communities, the present disclosure proposes counting the number of injectate microbial species detected in the produced fluid and using this number as a new metric for characterizing the relative connectivity of each production well with an injection well as well as how it changes over time.

To allow the above objective, the present disclosure provides the following solutions:

A method for characterizing inter-well connectivity is provided, including:
collecting an injected fluid microbial sample from an injection well and a produced fluid microbial sample from each corresponding production well in a selected well field;
extracting genomic DNA from the injected fluid microbial sample and the produced fluid microbial sample;
selecting a pair of universal primers for 16S rRNA gene amplicon sequencing, and subjecting the genomic DNA of the injected fluid microbial sample and the produced fluid microbial sample to 16S rRNA gene amplicon sequencing to obtain raw sequencing data of the injected fluid and raw sequencing data of each produced fluid;
inferring amplicon sequence variants (ASVs) from the raw sequencing data of the injected fluid microbial sample and the raw sequencing data of the produced fluid microbial sample to obtain an ASV table for the injected fluid microbial sample and an ASV table for the produced fluid microbial sample;
subjecting the ASV tables of an injection-production well pair to two-circle Venn diagram analysis to obtain a number of ASVs overlapped for the injection-production well pair, wherein the number of ASVs overlapped refers to a number of microbial species shared between the injected fluid microbial sample and the produced fluid microbial sample; and
determining inter-well connectivity for the injection-production well pair according to the number of ASVs overlapped in the injection-production well pair.

A computer device is provided, including a memory, a processor, and a computer program stored in the memory and executable on the processor, where the processor executes the computer program to implement steps of the method for characterizing inter-well connectivity described above.

A computer-readable storage medium is provided, where the computer-readable storage medium stores a computer program, and when executed by a processor, the computer program implements steps of the method for characterizing inter-well connectivity described above.

A computer program product is provided, including a computer program, where when executed by a processor, the computer program implements steps of the method for characterizing inter-well connectivity described above.

According to specific embodiments provided by the present disclosure, the present disclosure has the following technical effects:

The present disclosure provides a method for characterizing inter-well connectivity, a device, a medium, and a product. The method is as follows: a fluid microbial sample is collected from an injection well and each of the production wells in a selected well field and is subjected to 16S rRNA gene amplicon sequencing to obtain raw sequencing data; ASVs are inferred from the raw sequencing data to obtain ASV tables of the injected fluid and each of the produced fluids; and the ASV tables of each injection-production well pair is subjected to two-circle Venn diagram analysis to obtain the number of ASVs overlapped for each injection-production well pair, and the inter-well connectivity between each injection-production well pair is determined according to the corresponding numbers of ASVs overlapped. In the present disclosure, utilizing the fact that exogenous injection water and produced formation water often bear distinct microbial communities, the present disclosure uses the natural microbial community composition in exogenous injection water as a natural tracer, counts the number of injectate microbial species detected in the produced fluid to efficiently and conveniently characterize the relative connectivity of each production well with an injection well as well as how it changes over time. Compared with traditional tracer technology, the present disclosure does not require the injection of any chemical substance. Instead, the present disclosure utilizes the microbial signal in injected or produced fluids during reservoir injection-production operations that have not been utilized before. Therefore, the present disclosure is more eco-friendly than existing inter-well tracer technology, and does not cause any pollution to the formation.

### BRIEF DESCRIPTION OF THE DRAWINGS

To describe the technical solutions in the embodiments of the present disclosure or in the prior art clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a schematic flow chart of the method for characterizing inter-well connectivity provided in Example 1 of the present disclosure;

FIG. 2 is a schematic diagram of the definition of the number of ASVs overlapped (nASV-Overlap) index provided in Example 1 of the present disclosure;

FIG. 3 is a schematic diagram of characterizing the relative connectivities across multiple injection-production well pairs at multiple timepoints based on nASV-Overlap provided in Example 1 of the present disclosure; and

FIG. 4 is a diagram of an internal structure of a computer apparatus.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the embodiments are merely some rather than all of the embodiments of the present disclosure. All other embodiments obtained by a person of ordinary skill in the art based on the embodiments of the present disclosure without creative efforts shall fall within the protection scope of the present disclosure.

The objective of the present disclosure is to provide a method for characterizing inter-well connectivity, a device, a medium, and a product. Utilizing the fact that exogenous injection water and produced formation water often bear distinct microbial communities, the present disclosure efficiently and conveniently characterizes the relative connectivity across several production wells with an injection well, and monitors how such connectivity changes over time.

In order to make the above objective, features, and advantages of the present disclosure clear and comprehensible, the present disclosure will be further described in detail below in combination with accompanying drawings and specific implementations.

### Example 1

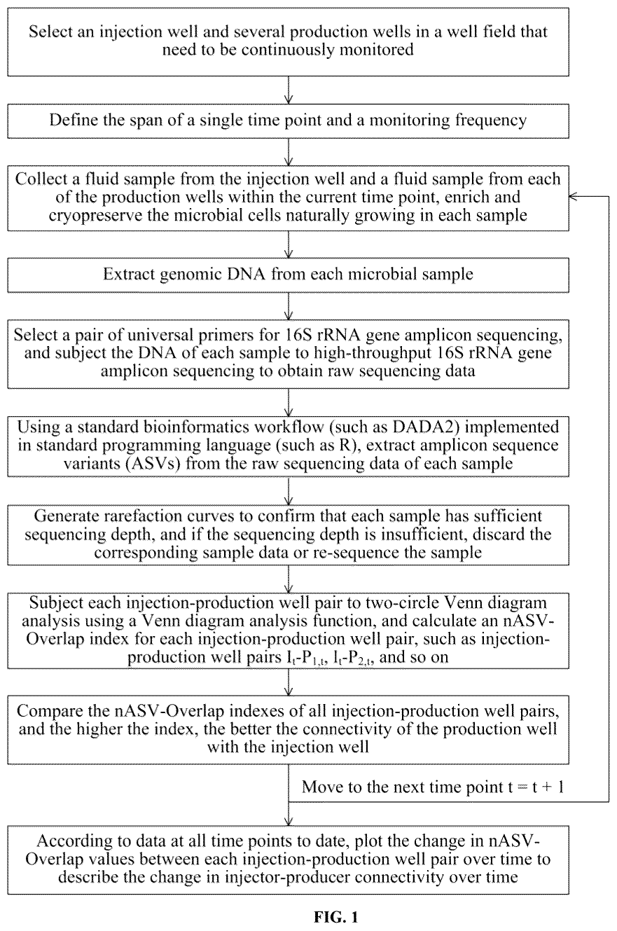

As shown in FIG. 1, a method for characterizing inter-well connectivity is provided in this example, specifically including the following steps:

Step 1: An injection well and several production wells that need to be continuously monitored in a well field are selected. For example, injection well I and production wells P₁, P₂, P₃, and P₄.

Step 2: The span of a single time point and a monitoring frequency are defined. For example, the span of a single time point is 2 d and the monitoring frequency is 1 time/week.

Step 3: A specified volume (such as 1 L to 10 L) of a fluid microbial sample is collected from the injection well and each production well within the current time point, including a microbial sample of the injected fluid and a microbial sample of the produced fluid from each production well. Microbial cells naturally growing in each sample are enriched and cryopreserved to ensure sample representativeness.

For example, the collection and storage of fluid microbial samples from injection well I and production wells P₁, P₂, P₃, and P₄, respectively, are completed within the first two days of week 1.

Step 4: Genomic DNA is extracted from each microbial sample using standard DNA extraction process and purified.

Step 5: Universal primers targeting the hypervariable regions of the 16S rRNA gene (such as the V4 and V5 hypervariable regions) are selected. Targeted region of the genomic DNA of each sample is amplified, and a sequencing library is constructed. The primers remain unchanged during the library construction at subsequent time points. Each microbial sample is subjected to 16S rRNA gene amplicon sequencing using common high-throughput sequencing platform (such as Illumina) to obtain raw data files (such as .fastq files), including raw sequencing data of the injected fluid and raw sequencing data of each produced fluid.

Step 6: Using a standard bioinformatics workflow (such as DADA2) implemented in standard programming language or software (such as R or Qiime), ASVs (also known as denoised DNA sequences) are extracted from the raw sequencing data to obtain an ASV table of each sample, including an ASV table of the injected fluid and an ASV table of each produced-fluid sample. An ASV is a denoised DNA sequence.

Step 7: Rarefaction curves are generated to confirm that each sample has a sufficient sequencing depth. If the sequencing depth is insufficient, the corresponding sample is discarded or re-sequencing sequencing is conducted.

In a rarefaction curve, the x-axis represents sequencing depth (i.e. the number of sequencing reads obtained for a sample) and the y-axis represents the number of unique ASVs observed. As sequencing depth increases, the number of unique ASVs (namely, the number of species) would first increase rapidly and then plateau. When the rarefaction curve plateaus, it means that all species in a sample have been adequately captured at the current sequencing depth, and that further increasing sequencing depth will not result in the detection of new ASVs. Therefore, when the actual sequencing depth of a sample has made the corresponding rarefaction curve reach a plateau, it means that the sequencing depth is sufficient. Conversely, when the actual sequencing depth does not make the corresponding rarefaction curve reach a plateau, that is, if the number of unique ASVs observed is still increasing, it means that the sequencing depth is not sufficient to capture all species in a sample, and that there are many existing species that remain undetected. In this case, the data point has limited representativeness and needs to be discarded or re-sequenced.

Step 8: The ASV tables of each injection-production well pair are subjected to two-circle Venn diagram analysis using a Venn diagram analysis function (such as the venn( ) function in eulerr package in the R language), and an nASV-Overlap index (namely, the number of ASVs overlapped) of each injection-production well pair is calculated. The connectivity of each production well with the injection well is determined based on the nASV-Overlap index.

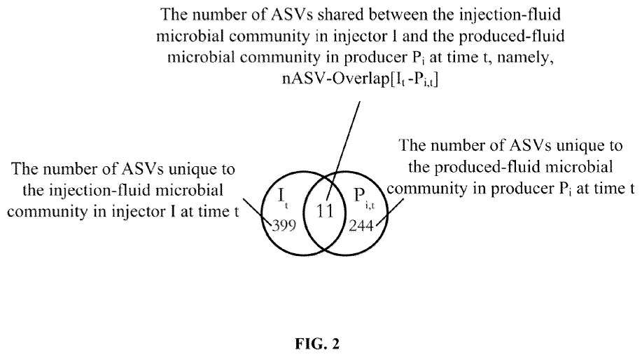

Utilizing the fact that exogenous injection water and produced formation water during reservoir injection-production operations often bear distinct microbial communities, DNA sequencing data of the microbial communities in injected-and produced-fluids are acquired by high-throughput sequencing and compared to search for injectate microbial DNA signal in the produced fluid. A novel “nASV-Overlap” metric is defined to quantify the number of microbial species shared between an injection-fluid sample and a produced-fluid sample. It is proposed that nASV-Overlap is positively correlated with the connectivity between an injection well and a production well. Therefore, the nASV-Overlap index can be used as a novel index to characterize the connectivity between an injection well and several production wells. The nASV-Overlap index is obtained through two-circle Venn diagram analysis on the injectate microbial community and each produced-fluid microbial community, as shown in FIG. 2. The nASV-Overlap index is defined as follows:

nASV-Overlap[Iₜ-P_{i,t}]=n(Iₜ)+n(P_{i,t})−n(Iₜ∪P_{i,t})=n(Iₜ∩P_{i,t}),

where Iₜ represents the unique ASV set of the injection well I at time t, obtained by sequencing the microbial community of the injection fluid; P_{i,t} represents the unique ASV set obtained by sequencing the produced fluid from the ith production well Pᵢ at time t; nASV-Overlap[Iₜ-P_{i,t}] represents the nASV-Overlap index between injection well I and production well Pᵢ at time t; n(Iₜ) and n(P_{i,t}) represent numbers of ASVs (equivalent to species richness) in Iₜ and P_{i,t}, respectively; n(Iₜ∪P_{i,t}) represents the number of ASVs in the union of Iₜand P_{i,t}; and n(Iₜ∩P_{i,t}) represents the number of ASVs in the intersection of Iₜ and P_{i,t}.

The present disclosure proposes that the probability for naturally-growing microbial cells in the injection fluid to successfully migrate to a production well is directly proportional to the connectivity of the production well with the injection well, and because microorganisms in an injection fluid exist in the form of a community made of many different species, the number of species in the injection fluid that successfully migrate to the production well is also directly proportional to the connectivity between the injection well and the production well. The number of injection-fluid species that reach the production well (namely, the nASV-Overlap value) can be obtained efficiently and conveniently by high-throughput sequencing in combination with two-circle Venn diagram analysis on the injected and produced fluids. Therefore, the nASV-Overlap value is proposed as a novel metric to characterize the relative connectivities across several production wells with an injection well as well as how they change over time.

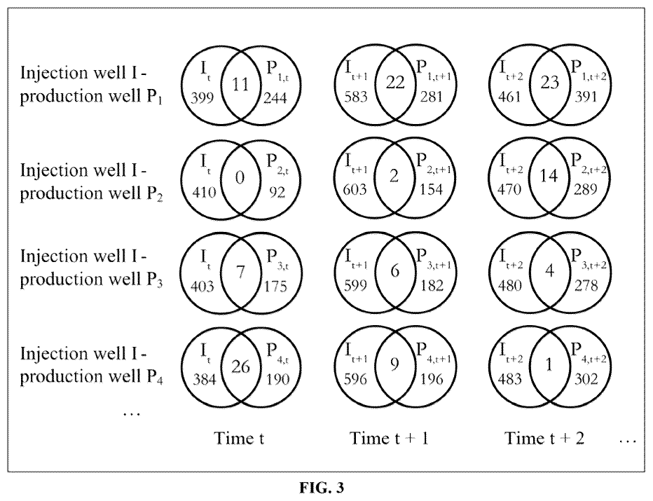

Step 9: nASV-Overlap indexes of all injection-production well pairs are compared. The higher the nASV-Overlap index, the better the connectivity with the injection well for a given production well, as shown in FIG. 3.

If nASV-Overlap[Iₜ-P_{4,t}]>nASV-Overlap[Iₜ-P_{1,t}]>nASV-Overlap[Iₜ-P_{3,t}]>nASV-Overlap[Iₜ-P_{2,t}], then the relative connectivity across each production well with the injection well at time t is as follows: P₄>P₁>P₃>P₂.

If nASV-Overlap[Iₜ₊₁-P_{1,t+1}]>nASV-Overlap[Iₜ₊₁-P_{4,t+1}]>nASV-Overlap[Iₜ₊₁-P_{3,t+1}]>nASV-Overlap[Iₜ₊₁-P_{2,t+1}], then the relative connectivity across each production well with the injection well at time t+1 is as follows: P₁>P₄>P₃>P₂.

If nASV-Overlap[Iₜ₊₂-P_{1,t+2}]>nASV-Overlap[Iₜ₊₂-P_{2,t+2}]>nASV-Overlap[Iₜ₊₂-P_{3,t+2}]>nASV-Overlap[Iₜ₊₂-P_{4,t+2}], then the relative connectivity across each production well with the injection well at time t+2 is as follows: P₁>P₂>P₃>P₄.

Step 10: Within the next time point, the sample collection and data analysis are repeated following the steps described above. For example, the collection and storage of fluid microbial samples from injection well I and production wells P₁, P₂, P₃, and P₄, respectively, are conducted once again within the first two days of week 2, and so on.

Step 11: According to data collected at all time points to date, the change in nASV-Overlap values between each injection-production well pair over time is plotted to describe the change of injector-producer connectivity over time, as shown in FIG. 3.

If nASV-Overlap[Iₜ₊₂-P_{1,t+2}]>nASV-Overlap[Iₜ₊₁-P_{1,t+1}]>nASV-Overlap[Iₜ-P_{1,t}], then the connectivity of production well P₁ with injection well I is as follows: time t+2>time t+1>time t.

If nASV-Overlap[Iₜ₊₂-P_{2,t+2}]>nASV-Overlap[Iₜ₊₁-P_{2,t+1}]>nASV-Overlap[Iₜ-P_{2,t}], then the connectivity of production well P₂ with injection well I is as follows: time t+2>time t+1>time t.

If nASV-Overlap[Iₜ-P_{3,t}]>nASV-Overlap[Iₜ₊₁-P_{3,t+1}]>nASV-Overlap[Iₜ₊₂-P_{3,t+2}], then the connectivity of production well P₃ with injection well I is as follows: time t>time t+1>time t+2.

If nASV-Overlap[Iₜ-P_{4,t}]>nASV-Overlap[Iₜ₊₁-P_{4,t+1}]>nASV-Overlap[Iₜ₊₂-P_{4,t+2}], then the connectivity of production well P₄ with injection well I is as follows: time t>time t+1>time t+2.

In this example, a technical workflow for characterizing inter-well connectivity based on the DNA of natural microbial communities in injected and produced fluids as well as the novel nASV-Overlap metric is established. This workflow is conducive to the comprehensive specification and quality control of the present disclosure when being used and promoted. The following quality control steps in the workflow is proposed and their necessity is emphasized: 1) The definition of the span of each single time point should be clear and as consistent as possible. 2) Each set of injected-and produced-fluid samples should be collected within the span of each time point and should be cryopreserved as soon as possible to ensure sample representativeness. This ensures that the set of samples is representative of the state of reservoir interwell connectivity at the current time point, and that reduced accuracy of connectivity characterization caused by potential injection-fluid community fluctuation is avoided. 3) After sequencing is completed, the raw data should undergo quality control, and in particular, sufficient sequencing depth should be confirmed. Data that does not meet the quality control requirements should be discarded or re-sequenced.

In this example, compared with existing artificial tracer technology, the present disclosure does not require the addition of any chemical substances to the formation water. Instead, utilizing the natural contrast in injection-and production-fluid microbial community composition caused by their distinct source environmental conditions, the present disclosure uses high-throughput sequencing combined with Venn diagram analysis to detect injection-fluid microbial signal in the production fluid to characterize injector-producer interwell connectivity. Advantages are as follows:
1. Existing tracer technology requires high-frequency sampling at each production well to obtain a complete breakthrough curve (generally, 30 to 50 data points hence 30 to 50 samples are needed per injector-producer well pair), so as to calculate a tracer recovery rate for each production well by integrating over the breakthrough curve. The connectivity across each production well with the injection well is determined based on the tracer recovery rate (the higher the recovery rate, the better the connectivity of a production well with the injection well). In comparison, the present disclosure only requires the collection of one sample from the injected fluid and one sample from each produced fluid (two samples total per injector-producer well pair), such that the nASV-Overlap value between the sample pair can be obtained through high-throughput sequencing and the connectivity between each injection-production well pair can be determined. The present disclosure greatly reduces sampling frequency and thus is more cost-effective and efficient.
2. Traditional tracer method often fails during field tests because it is difficult to capture the tracer plume in a production well. The present disclosure does not require the capture of any tracer plume, therefore the success rate of field tests is high.
3. Because of its low cost and high efficiency, when the connectivity between the injection well and each production well changes dynamically over time, the present disclosure is easily repeated at the next time point. In terms of characterizing inter-well connectivity, the information content of one nASV-Overlap data point is equivalent to the information content of an entire chemical tracer breakthrough curve (typically including 30 to 50 data points). Therefore, the present disclosure allows the long-term monitoring of injector-producer connectivity efficiently and cost-effectively, which is not achievable by traditional tracer method.
4. Based on the above-mentioned capability for the long-term monitoring of injector-producer connectivity, the nASV-Overlap values across different producers with the injector can be cross compared in order to determine whether the injectate uptake is synergistic or competitive across different producers. This is not realizable by existing tracer technology.

### Example 2

A computer device is provided, including a memory, a processor, and a computer program stored in the memory and executable on the processor. The processor executes the computer program to implement steps of the method for characterizing inter-well connectivity in Example 1.

### Example 3

A computer-readable storage medium is provided. The computer-readable storage medium stores a computer program, and when executed by a processor, the computer program implements steps of the method for characterizing inter-well connectivity in Example 1.

### Example 4

A computer program product is provided, including a computer program. When executed by a processor, the computer program implements steps of the method for characterizing inter-well connectivity in Example 1.

### Example 5

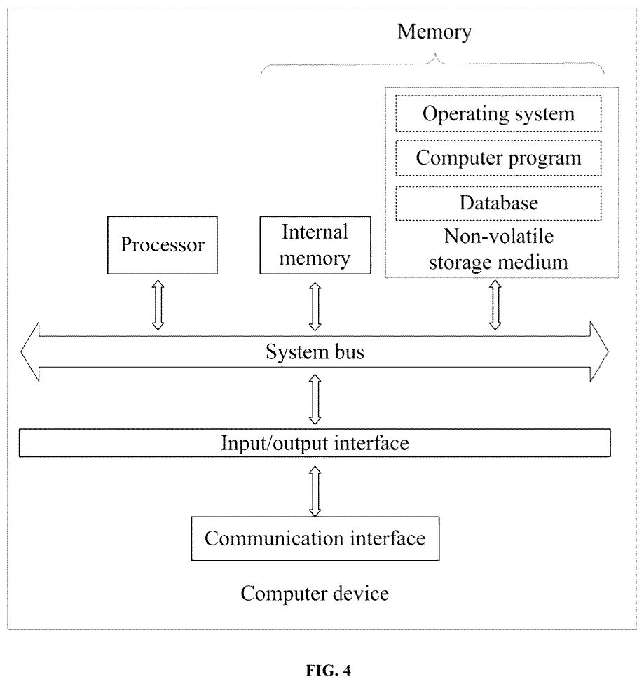

A computer apparatus is provided. The computer apparatus may be a database and can have an internal structure shown in FIG. 4. The computer apparatus includes a processor, a memory, an input/output interface (I/O), and a communication interface. The processor, the memory, and the input/output interface are connected through a system bus, and the communication interface is connected to the system bus through the input/output interface. The processor of the computer apparatus is configured to provide computing and control capabilities. The memory of the computer apparatus includes a non-volatile storage medium and an internal memory. The non-volatile storage medium stores an operating system, a computer program, and a database. The internal memory provides an environment for operations of the operating system and the computer program in the non-volatile storage medium. The database of the computer apparatus is configured to store pending transactions. The input/output interface of the computer apparatus is configured to exchange information between the processor and an external apparatus. The communication interface of the computer apparatus is configured to communicate with an external terminal through a network. When executed by the processor, the computer program implements the method for characterizing inter-well connectivity in Example 1.

It should be noted that the object information (including, but not limited to, object apparatus information, object personal information, or the like) and data (including, but not limited to, data for analysis, stored data, displayed data, or the like) involved in the present disclosure all are information and data authorized by an object or fully authorized by all parties, and the acquisition, use, and processing of relevant data need to comply with the relevant laws, regulations, and standards of relevant countries and regions.

Those of ordinary skill in the art may understand that all or some of the procedures in the method of the above embodiments may be implemented by a computer program instructing related hardware. The computer program may be stored in a non-volatile computer-readable storage medium. When the computer program is executed, the procedures in the embodiments of the above method may be implemented. Any reference to a memory, a database, or other media used in the embodiments of the present disclosure may include at least one of non-volatile and volatile memories. Non-volatile memories may include a read-only memory (ROM), a magnetic tape, a floppy disk, a flash memory, an optical memory, a high-density embedded non-volatile memory, a resistive random access memory (ReRAM), a magnetoresistive random access memory (MRAM), a ferroelectric random access memory (FRAM), a phase change memory (PCM), a graphene memory, or the like. Volatile memories may include a random access memory (RAM) or an external cache memory. As an illustration rather than a limitation, the RAM may be in various forms, such as a static random access memory (SRAM) or a dynamic random access memory (DRAM). The database involved in each embodiment provided by the present disclosure may include at least one selected from the group consisting of a relational database and a non-relational database. The non-relational database can include a block chain-based distributed database, but is not limited thereto. The processor involved in each embodiment provided by the present disclosure may be a general-purpose processor, a central processor, a graphic processor, a digital signal processor, a programmable logic device, or a quantum computing-based data processing logic device, and is not limited thereto.

The technical characteristics of the above embodiments can be arbitrarily combined. For brevity of description, not all possible combinations of the technical characteristics of the above embodiments are described. However, these combinations of the technical characteristics should be construed as falling within the scope defined by the specification as long as there is no contradiction among the combinations.

Specific examples are used herein to explain the principles and implementations of the present disclosure. The description of the examples is merely intended to help understand the method of the present disclosure and its core ideas. In addition, those of ordinary skill in the art can make various modifications to the specific implementations and application scope in accordance with the teachings of the present disclosure. In conclusion, the content of the present specification shall not be construed as a limitation to the present disclosure.

1. A method for characterizing inter-well connectivity, comprising:
collecting an injected fluid microbial sample from an injection well and a produced fluid microbial sample from each corresponding production well in a selected well field;
extracting genomic DNA from the injected fluid microbial sample and the produced fluid microbial sample;
selecting a pair of universal primers for 16S rRNA gene amplicon sequencing, and subjecting the genomic DNA of the injected fluid microbial sample and the produced fluid microbial sample to 16S rRNA gene amplicon sequencing to obtain raw sequencing data of the injected fluid and raw sequencing data of each produced fluid;
extracting amplicon sequence variants (ASVs) from the raw sequencing data of the injected fluid microbial sample and the raw sequencing data of the produced fluid microbial sample to obtain an ASV table for the injected fluid microbial sample and an ASV table for the produced fluid microbial sample; wherein the ASV tables for the injected fluid microbial sample and the produced fluid microbial sample comprise denoised DNA sequence data of the injected fluid microbial sample and denoised DNA sequence data of the produced fluid microbial sample, respectively;
performing two-circle Venn diagram analysis on the ASV tables for the injected fluid microbial sample and the produced fluid microbial sample to obtain a number of ASVs overlapped, wherein the number of ASVs overlapped refers to a number of microbial species that are common to both the injected fluid microbial sample and the produced fluid microbial sample;
wherein a calculation formula for the number of ASVs overlapped is as follows:
nASV-Overlap[Iₜ-P_{i,t}]=n(IₜP_{i,t}).
wherein nASV-Overlap[Iₜ-P_{i,t}] represents a number of ASVs overlapped between an injection well I and a production well Pᵢ at time t; Iₜ represents a unique ASV set of injection fluid in the injection well I at time t; P_{i,t} represents a unique ASV set of produced fluid from an ith production well Pᵢ at time t; and n(Iₜ∩P_{i,t}) represents a number of ASVs in an intersection of Iₜ and P_{i,t};
determining inter-well connectivity between the injection well and the corresponding production well according to the number of ASVs overlapped; wherein the number of ASVs overlapped is proportional to the inter-well connectivity: and
based on the inter-well connectivity, building a reservoir model for forecasting production of underground resources.

2. The method for characterizing inter-well connectivity according to claim 1, wherein before performing two-circle Venn diagram analysis on the ASV tables for the injected fluid microbial sample and the produced fluid microbial sample, the method further comprises:
generating rarefaction curves according to the ASV tables of the injected fluid microbial sample and the produced fluid microbial sample, respectively;
determining whether a sequencing depth of each microbial sample is sufficient according to the rarefaction curve, wherein when the rarefaction curve levels off or reaches a plateau, sequencing depth is considered to be sufficient, and the microbial sample comprises the microbial sample of the injected fluid and the microbial sample of the produced fluid from the corresponding production well;
if sequencing depth is sufficient, conducting two-circle Venn diagram analysis; and
if sequencing depth is not sufficient, discarding the corresponding microbial sample or subjecting the microbial sample with insufficient sequencing depth to 16S rRNA gene amplicon sequencing once again.

3. The method for characterizing inter-well connectivity according to claim 1, wherein after the determining inter-well connectivity for an injection-production well pair according to the number of ASVs overlapped in the injection-production well pair is implemented, the method further comprises:
obtaining numbers of ASVs overlapped at all time points for the injection-production well pair;
plotting temporal variations in the number of ASVs overlapped between each injection-production well pair; and
according to the temporal variations, acquiring patterns of change in injector-producer connectivities over time.

4. (canceled)

5. The method for characterizing inter-well connectivity according to claim 1, wherein the 16S rRNA gene amplicon sequencing is conducted with a high-throughput sequencing platform.

6. A computer device, comprising a memory, a processor, and a computer program stored in the memory and executable on the processor, wherein the processor executes the computer program to implement steps of the method for characterizing inter-well connectivity according to claim 1.

7. A computer-readable storage medium, wherein the computer-readable storage medium stores a computer program, and when executed by a processor, the computer program implements steps of the method for characterizing inter-well connectivity according to claim 1.

8. A computer program product, comprising a computer program, wherein when executed by a processor, the computer program implements steps of the method for characterizing inter-well connectivity according to claim 1.

9. The computer device according to claim 6, wherein before performing two-circle Venn diagram analysis on the ASV tables for the injected fluid microbial sample and the produced fluid microbial sample, the method further comprises:
generating rarefaction curves according to the ASV tables of the injected fluid microbial sample and the produced fluid microbial sample, respectively;
determining whether a sequencing depth of each microbial sample is sufficient according to the rarefaction curve, wherein when the rarefaction curve levels off or reaches a plateau, sequencing depth is considered to be sufficient, and the microbial sample comprises the microbial sample of the injected fluid and the microbial sample of the produced fluid from the corresponding production well;
if sequencing depth is sufficient, conducting two-circle Venn diagram analysis; and
if sequencing depth is not sufficient, discarding the corresponding microbial sample or subjecting the microbial sample with insufficient sequencing depth to 16S rRNA gene amplicon sequencing once again.

10. The computer device according to claim 6, wherein after the step of determining inter-well connectivity for an injection-production well pair according to the number of ASVs overlapped in the injection-production well pair is implemented, the method further comprises:
obtaining numbers of ASVs overlapped at all time points for the injection-production well pair;
plotting temporal variations in the number of ASVs overlapped between each injection-production well pair; and
according to the temporal variations, acquiring patterns of change in injector-producer connectivities over time.

11. (canceled)

12. The computer device according to claim 6, wherein the 16S RNA gene amplicon sequencing is conducted with a high-throughput sequencing platform.

13. The computer-readable storage medium according to claim 7, wherein before performing two-circle Venn diagram analysis on the ASV tables for the injected fluid microbial sample and the produced fluid microbial sample, the method further comprises:
generating rarefaction curves according to the ASV tables of the injected fluid microbial sample and the produced fluid microbial sample, respectively;
determining whether a sequencing depth of each microbial sample is sufficient according to the rarefaction curve, wherein when the rarefaction curve levels off or reaches a plateau, sequencing depth is considered to be sufficient, and the microbial sample comprises the microbial sample of the injected fluid and the microbial sample of the produced fluid from the corresponding production well;
if sequencing depth is sufficient, conducting two-circle Venn diagram analysis; and
if sequencing depth is not sufficient, discarding the corresponding microbial sample or subjecting the microbial sample with insufficient sequencing depth to 16S rRNA gene amplicon sequencing once again.

14. The computer-readable storage medium according to claim 7, wherein after the step of determining inter-well connectivity for an injection-production well pair according to the number of ASVs overlapped in the injection-production well pair is implemented, the method further comprises:
obtaining numbers of ASVs overlapped at all time points for the injection-production well pair;
plotting temporal variations in the number of ASVs overlapped between each injection-production well pair; and
according to the temporal variations, acquiring patterns of change in injector-producer connectivities over time.

15. (canceled)

16. The computer-readable storage medium according to claim 7, wherein the 16S rRNA gene amplicon sequencing is conducted with a high-throughput sequencing platform.

17. The computer program product according to claim 8, wherein before performing two-circle Venn diagram analysis on the ASV tables for the injected fluid microbial sample and the produced fluid microbial sample, the method further comprises:
generating rarefaction curves according to the ASV tables of the injected fluid microbial sample and the produced fluid microbial sample, respectively;
determining whether a sequencing depth of each microbial sample is sufficient according to the rarefaction curve, wherein when the rarefaction curve levels off or reaches a plateau, sequencing depth is considered to be sufficient, and the microbial sample comprises the microbial sample of the injected fluid and the microbial sample of the produced fluid from the corresponding production well;
if sequencing depth is sufficient, conducting two-circle Venn diagram analysis; and
if sequencing depth is not sufficient, discarding the corresponding microbial sample or subjecting the microbial sample with insufficient sequencing depth to 16S rRNA gene amplicon sequencing once again.

18. The computer program product according to claim 8, wherein after the step of determining inter-well connectivity for an injection-production well pair according to the number of ASVs overlapped in the injection-production well pair is implemented, the method further comprises:
obtaining numbers of ASVs overlapped at all time points for the injection-production well pair;
plotting temporal variations in the number of ASVs overlapped between each injection-production well pair; and
according to the temporal variations, acquiring patterns of change in injector-producer connectivities over time.

19. (canceled)

20. The computer program product according to claim 8, wherein the 16S rRNA gene amplicon sequencing is conducted with a high-throughput sequencing platform.
